(19) United States
(12) Patent Application Publication
(10) Pub. No.: US 2021/0340115 A1
(43) Pub. Date: Nov. 4, 2021

(54) A PROCESS FOR THE PREPARATION OF OZANIMOD AND ITS INTERMEDIATE (S)-l-AMINO-2,3-DIHYDRO-1H-INDENE-4-CARBONITRILE

(71) Applicant: PHARMAZELL GMBH, Raubling (DE)

(72) Inventors: Kai DONSBACH, Raubling (DE); Jayprakash Ajitsingh PARIHAR, Parawada, Visakhapatnam (IN); Sridhar PRATHA, Parawada, Visakhapatnam (IN); Chinnayya Setty SATYAVARAPU, Parawada, Visakhapatnam (IN); Leela Kumar NALLURI, Parawada, Visakhapatnam (IN); Prasad MATTURTI, Parawada, Visakhapatnam (IN)

(21) Appl. No.: 17/274,535

(22) PCT Filed: Sep. 12, 2019
(86) PCT No.: PCT/EP2019/074348

§ 371 Date: Mar. 9, 2021

(30) Foreign Application Priority Data
Sep. 12, 2018 (IN) 201841034453

(51) Int. Cl. C07D 271/06

(52) U.S. Cl. CPC C07D 271/06; C07B 2200/07

(57) ABSTRACT

The present invention relates to an improved process for preparation of Ozanimod (I) or pharmaceutically acceptable salts thereof. The present invention also relates to an improved process for preparation of (S)-1-amino-2,3-dihydro-1H-indene-4-carbonitrile (II) or its optically active acid salts.

### FIELD OF INVENTION

The present invention relates to an improved process for preparation of Ozanimod (I) or pharmaceutically acceptable salts thereof.

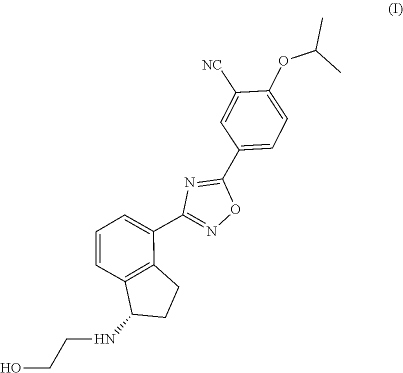 [SMILES: CC(C)Oc1ccc(-c2nc(-c3cccc4c3CC[C@@H]4NCCO)no2)cc1C#N]

The present invention also relates to an improved process for preparation of (S)-1-amino-2,3-dihydro-1H-indene-4-carbonitrile (II) or its optically active acid salts.

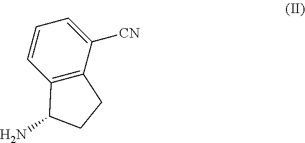 [SMILES: N#Cc1cccc2c1CC[C@@H]2N]

The formula (II) is a key intermediate in the preparation of Ozanimod of formula (I).

### BACKGROUND OF INVENTION

Ozanimod (I) is chemically known as 5-[3-[(1S)-2,3-dihydro-1-[(2-hydroxyethyl)amino]-1H-inden-4-yl]-1,2,4-oxadiazol-5-yl]-2-(1-methylethoxy)-benzonitrile.

Ozanimod is a novel oral selective S1PR (Sphingosine-1-phosphate receptor) modulator developed by Receptos for the treatment of autoimmune diseases, especially for the treatment of multiple sclerosis and ulcerative colitis. In clinical trials, Ozanimod's clinical results showed better safety than fingolimod, especially in terms of cardiac safety. Based on the promising pharmacokinetic, pharmacodynamic and safety data of Ozanimod, it can meet a differentiated development strategy and is expected to become an important second-generation S1PR modulator drug. The chemical structure of the drug is represented by formula (I).

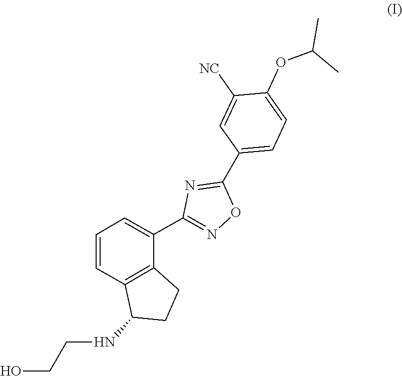 [SMILES: CC(C)Oc1ccc(-c2nc(-c3cccc4c3CC[C@@H]4NCCO)no2)cc1C#N]

Ozanimod is disclosed in WO 2011/060392 A1. WO '392 A1 also discloses a process for the preparation of Ozanimod (I) or its pharmaceutically acceptable salts thereof, by reacting (S)-1-amino-2,3-dihydro-1H-indene-1-yl)-4-carbonitrile or its salt (II) with Boc anhydride and triethyl amine (TEA) in dichloromethane (DCM) to produce (S)-tert-butyl-4-cyano-2,3-dihydro-1H-inden-1-yl-carbamate of formula (III).

Compound of formula (III) is reacted with (2-bromoethoxy)(tert-butyl)dimethylsilane in presence of sodium hydride (NaH) in N,N-dimethylformamide (DMF) to produce (S)-tert-butyl-2-(tert-butyldimethylsilyloxy)ethyl(4-cyano-2,3-dihydro-1H-inden-1-yl)carbamate of formula (IV), which is further treated with hydroxylamine in presence of triethylamine and ethanol to produce (S)-tert-butyl-2-(tert-butyldimethylsilyloxy)ethyl(4-(N-hydroxycarbamimidoyl)-2,3-dihydro-1H-inden-1-yl)carbamate of formula (V).

(S)-tert-butyl-2-(tert-butyldimethylsilyloxy)ethyl(4-(N-hydroxycarbamimidoyl)-2,3-dihydro-1H-inden-1-yl)carbamate of formula (V) is reacted with 3-cyano-4-isopropoxybenzoic acid in presence of hydroxybenzotriazole to produce a mixture of (S)-tert-butyl-2-(tert-butyldimethylsilyloxy)ethyl (4-(5-(3-cyano-4-isopropoxyphenyl)-1,2,4-oxadiazol-3-yl)-2,3-dihydro-1H-inden-1-yl)carbamate of formula (VI) and (S)-tert-butyl(4-(5-(3-cyano-4-isopropoxyphenyl)-1,2,4-oxadiazol-3-yl)-2,3-dihydro-1H-inden-1-yl)(2-hydroxyethyl)carbamate of formula (VII), which is further hydrolysed with HCl/dioxane to yield Ozanimod (I).

The route of synthesis is shown in scheme-I:

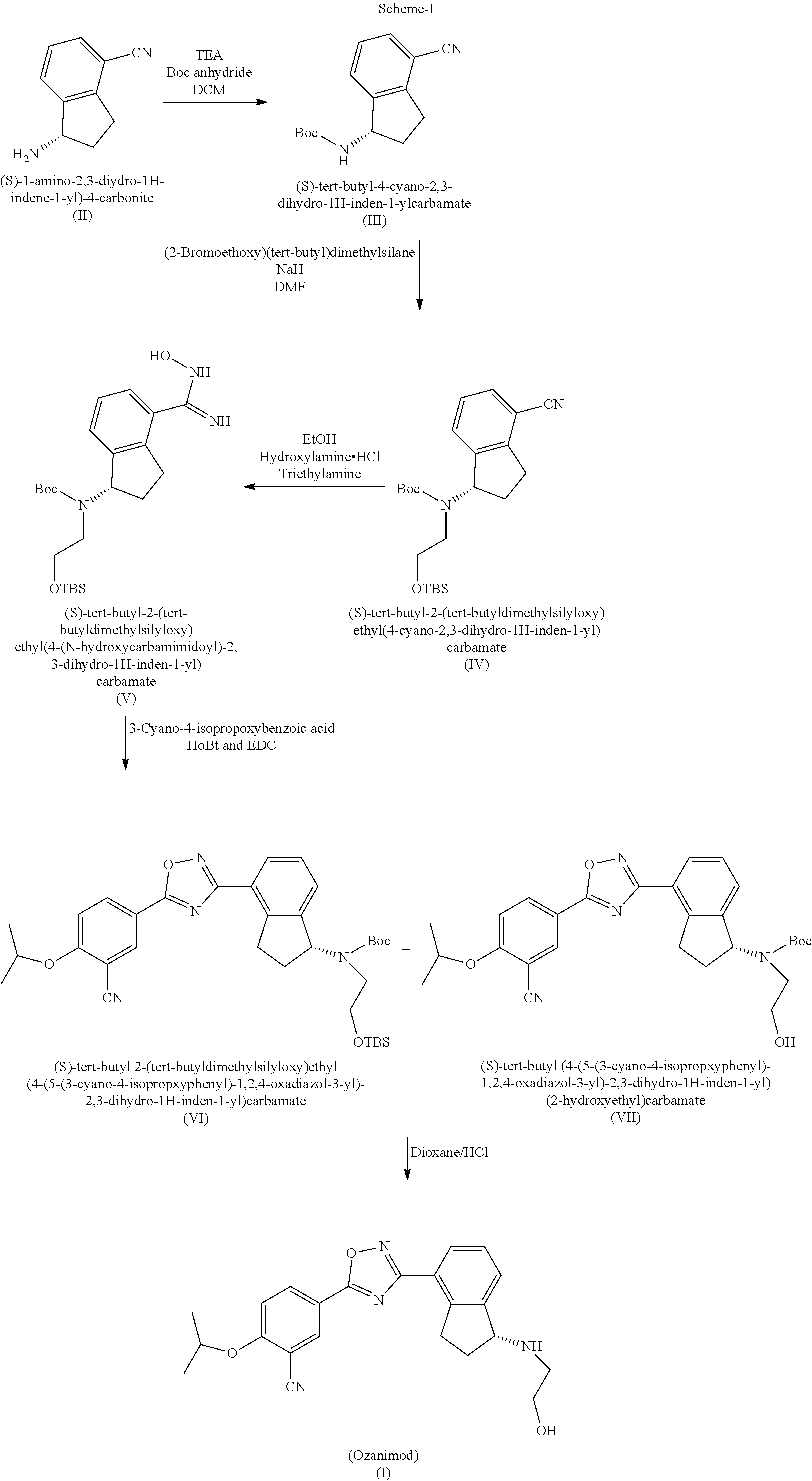

The major disadvantage with the above process is that it involves usage of sodium hydride which is highly pyrophoric and difficult to handle on large scale. Further (2-bromoethoxy)(tert-butyl)dimethylsilane is expensive and not readily commercially available.

Therefore, an alternative process is beneficiary which for example, involves reagents that are less expensive and/or easier to handle, consume smaller amounts of reagents, provide a higher yield of product, have smaller and/or more eco-friendly waste products, and/or provide a product of higher purity.

(S)-1-Amino-2,3-dihydro-1H-indene-4-carbonitrile (II) or its salt is a key intermediate in the preparation of Ozanimod of Formula (I).

WO '392 A1 also discloses a process for the preparation of (S)-1-amino-2,3-dihydro-1H-indene-4-carbonitrile (II) or its salt by reacting 1-oxo-2,3-dihydro-1H-indene-4-carbonitrile of formula (VIII) with (S)-2-methylpropane-2-sulfinamide in toluene to produce (S)—N-(4-cyano-2,3-dihydro-1H-indene-1-ylidene)-2-methylpropane-2-sulfinamide of formula (IX), which is further undergoes reduction with NaBH₄ at −78° C. to produce (S)—N-(4-cyano-2,3-dihydro-1H-inden-1-yl)-2-methylpropane-2-sulfinamide of formula (X). Further (S)—N-(4-cyano-2,3-dihydro-1H-inden-1-yl)-2-methylpropane-2-sulfinamide of formula (X) treated with HCl in methanol to produce (S)-1-amino-2,3-dihydro-1H-indene-4-carbonitrile of formula (II).

The route of synthesis is shown in scheme-II:

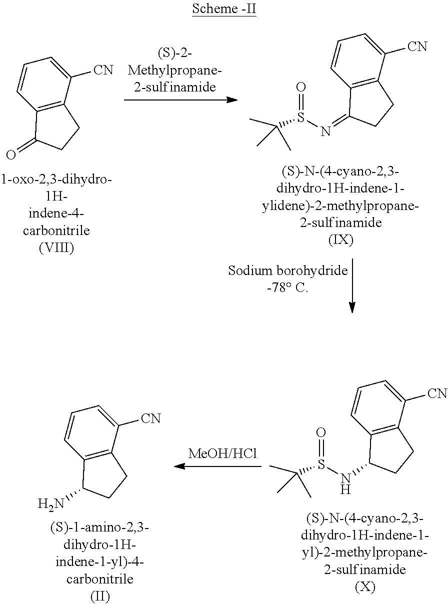 [SMILES: N#Cc1cccc2c1CCC2=O ; CC(C)(C)[S@](=O)/N=C1/CCc2c(C#N)cccc21 ; N#Cc1cccc2c1CC[C@@H]2N ; CC(C)(C)[S@](=O)N[C@H]1CCc2c(C#N)cccc21]

The major disadvantage of above process is that the above process involves the usage of an expensive chiral auxiliary and cryogenic condition. The synthetic process for formula (II) requires several steps. It is commercially beneficiary to reduce the number of chemical steps.

Hence, there is a desire to develop a more cost effective and commercially viable process for the preparation of Ozanimod (I) and its key intermediate (S)-1-amino-2,3-dihydro-1H-indene-4-carbonitrile (II) or its salts.

The present invention relates to a process for the preparation of Ozanimod (I) by reacting (S)-5-(3-(1-amino-2,3-dihydro-1H-inden-4-yl)-1,2,4-oxadiazol-5-yl)-2-isopropoxy-benzonitrile or its salt with alkyl-2-haloacetate in the presence of a suitable base followed by reduction to produce Ozanimod (I) or its salts.

The present invention also relates to a process for the preparation of pure (S)-1-amino-2,3-dihydro-1H-indene-4-carbonitrile (II) or its salt by reacting (R,S)-1-amino-2,3-dihydro-1H-indene-4-carbonitrile using optically active acid.

### OBJECT OF INVENTION

The main embodiment of the present invention is to provide a simple, cost effective process for the preparation of Ozanimod (I) with high purity and good yield on commercial scale.

Another embodiment of the present invention provides, (S)-1-amino-2,3-dihydro-1H-indene-4-carbonitrile di-p-toluyl-L-tartaric acid salt, a process for its preparation with high purity and good yield on commercial scale.

Another embodiment of the present invention provides, (S)-1-amino-2,3-dihydro-1H-indene-4-carbonitrile or its optically active acid salt, a process for its preparation with high purity and good yield on commercial scale.

Another embodiment of the present invention provides (S)-1-amino-2,3-dihydro-1H-indene-4-carbonitrile salt for its preparation and its conversion to Ozanimod (I).

### SUMMARY OF THE INVENTION

Accordingly, in one embodiment, the present invention provides (S)-1-amino-2,3-dihydro-1H-indene-4-carbonitrile di-p-toluyl-L-tartaric acid salt (IIb).

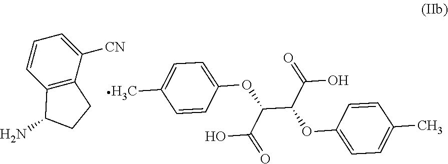

In another embodiment, the present invention provides, a process for the preparation of (S)-1-amino-2,3-dihydro-1H-indene-4-carbonitrile di-p-toluyl-L-tartaric acid salt (IIb): which comprises, reacting (R,S)-1-amino-2,3-dihydro-1H-indene-1-yl)-4-carbonitrile (Racemic II)

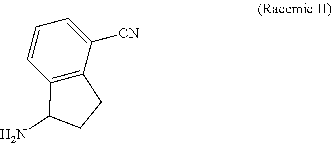 [SMILES: N#Cc1cccc2c1CCC2N]

with di-p-toluyl-L-tartaric acid salt in a presence of a solvent to produce (S)-1-amino-2,3-dihydro-1H-indene-4-carbonitrile di-p-toluyl-L-tartaric acid salt (IIb).

In another embodiment, the present invention provides, an improved process for the preparation of (S)-1-amino-2,3-dihydro-1H-indene-4-carbonitrile (II),

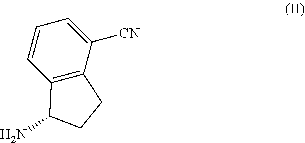 [SMILES: N#Cc1cccc2c1CC[C@@H]2N]

which comprises:
(i) reacting (R,S)-1-amino-2,3-dihydro-1H-indene-1-yl)-4-carbonitrile (Racemic II) with optically active acid to produce (S)-1-amino-2,3-dihydro-1H-indene-4-carbonitrile optically active acid salt (IIa),

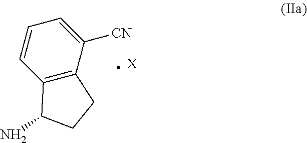

(ii) converting the compound (Ha) to (S)-1-amino-2,3-dihydro-1H-indene-4-carbonitrile (II).

wherein X is optically active acid.

In another embodiment, the present invention also provides a process for the preparation of Ozanimod (I):

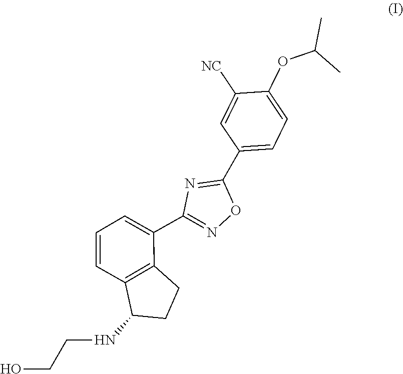 [SMILES: CC(C)Oc1ccc(-c2nc(-c3cccc4c3CC[C@@H]4NCCO)no2)cc1C#N]

which comprises:
i) reacting compound of formula (XI) or its salt,

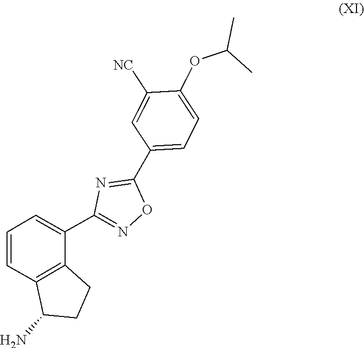 [SMILES: CC(C)Oc1ccc(-c2nc(-c3cccc4c3CC[C@@H]4N)no2)cc1C#N]

with a 2-haloacetate compound, as for example an alkyl-2-haloacetate, of formula (XII)

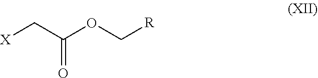

wherein, X represents halo compound and consist of Cl, Br or I; R is selected from the group consisting of H; C₁ to C₈ alkyl, in particular C₁ to C₄ alkyl; arylalkyl, wherein alkyl is C₁ to C₈ alkyl, in particular C₁ to C₄ alkyl; aryl; and heteroaryl; in the presence of suitable base to produce compound of formula (XIII).

“Alkyl” in the context of the invention represents straight-chain or branched hydrocarbon radicals having 1 to 8, 1 to 7, 1 to 6, or 1 to 4, carbon atoms, as for example methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl; n-heptyl, and n-octyl and the singly or multiply branched analogues thereof.

“Aryl” in the context of the invention represents mono- or polycyclic, preferably mono- or bicyclic, optionally substituted aromatic radicals having 6 to 20, for example 6 to 10, ring carbon atoms, for example phenyl, biphenyl, naphthyl such as 1- or 2-naphthyl, tetrahydronaphthyl, fluorenyl, indenyl and phenanthrenyl. These aryl radicals may optionally bear 1, 2, 3, 4, 5 or 6 identical or different substituents.

“Substituents” for radicals specified herein are especially, unless stated otherwise, selected from keto groups, —COOH, —COO-alkyl, —OH, —SH, —CN, amino, —NO₂, or alkyl groups.

“Heteroaryl” in the context of the invention represents:
5-membered aromatic heterocyclyl residues comprising, besides carbon atoms, 1 to 4 nitrogen, sulfur and/or oxygen atom as ring members, e.g. 2-furyl, 3 furyl, 2-thienyl, 3 thienyl, 2-oxazolyl, 4 oxazolyl, 5-oxazolyl, 2-thiazolyl, 4-thiazolyl, 5-thiazolyl, 2-imidazolyl, 4-imidazolyl, 3- or 4-isoxazolyl, 3- or 4-isothiazolyl, 1,2,4 thiadiazol-3-yl, 1,2,4-thiadiazol-5-yl, 1,2,4 oxadiazol-3-yl, 1,2,4-oxadiazol-5-yl and 1,3,4-oxadiazol-2-yl; 1-, 2- or 3-pyrrolyl, 1-, 3- or 4-pyrazolyl, 1-, 2- or 4-imidazolyl, 1,2,3-triazol-1-yl, 1,2,3-triazol-2-yl, 1,2,3-triazol-4-yl, 1,2,3-triazol-5-yl, 1,2,3-triazol-4-yl, 1,2,4-triazol-1-yl, 1,2,4-triazol-3-yl, 1,2,4-triazol-5-yl, 1,2,4-triazol-4-yl, 1,2,4-triazol-3-yl, 1,3,4-triazol-2-yl; tetrazol-1-yl, tetrazol-5-yl, tetrazol-2-yl and tetrazol-5-yl;
6-membered aromatic heterocyclyl residues comprising, besides carbon atoms, 1 to 3 nitrogen atoms as ring members, e.g. 2 pyridinyl, 3-pyridinyl, 4-pyridinyl, 3-pyridazinyl, 4-pyridazinyl, 2-pyrimidinyl, 4 pyrimidinyl, 5-pyrimidinyl, 2-pyrazinyl, 1,2,4-triazin-3-yl; 1,2,4-triazin-5-yl, 1,2,4-triazin-6-yl and 1,3,5-triazin-2-yl.
polynuclear, such as e.g. di- or trinuclear, cyclic ring systems in which one of the aforementioned mononuclear heteroaryl radicals is condensed with at least one further identical or different heteroaryl ring, at least one aryl ring, in each case in accordance with the above definition, and/or at least one saturated or mono- or polyunsaturated, as for example mono- or di-unsaturated, cycloaliphatic ring having 4, 5, 6, 7 or 8 ring carbon atoms.

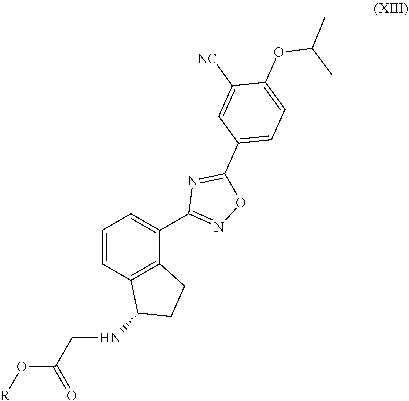

(iii) reacting the compound of formula (XIII) with a reducing agent to produce Ozanimod of formula (I).
(iv) optionally, converting Ozanimod of formula (I) to its pharmaceutically acceptable salts thereof.

In another embodiment, the present invention provides the use of (S)-1-amino-2,3-dihydro-1H-indene-4-carbonitrile (II) or its salt prepared by present invention in the preparation of Ozanimod of formula (I).

### DETAILED DESCRIPTION OF THE INVENTION

In one embodiment, an improved process for the preparation of (S)-1-amino-2,3-dihydro-1H-indene-4-carbonitrile di-p-toluyl-L-tartaric acid salt (IIb).

The reaction comprises, reacting of (R,S)-1-amino-2,3-dihydro-1H-indene-4-carbonitrile or its salt (Racemic II) using di-p-toluyl-L-tartaric acid salt in presence of a solvent to produce (S)-1-amino-2,3-dihydro-1H-indene-4-carbonitrile di-p-toluyl-L-tartaric acid salt (IIb).

The reaction is carried out at temperature below 75° C., preferably below 50° C., more preferably below 35° C. The salt formation can be carried out in a solvent selected from a hydrocarbon, as for example aliphatic or aromatic solvents, like for example pentane, hexane, cyclohexane, heptane, methylcyclohexane (MCH), toluene and xylene etc. or mixtures thereof; or a cyclic or noncyclic ether, like non-cyclic di-C₁-C₄-alkyl ethers, as for example methyl-t-butyl ether, diethyl ether, dibutyl ether, diisopropyl ether, cyclic ethers, like 1,4-dioxane or tetrahydrofuran or mixtures thereof; or a ketone solvent , like di-C₁-C₄-alkyl ketones, as for example acetone, butanone, methyl ethyl ketone, methyl isobutyl ketone, diisobutyl ketone, methyl isopropyl ketone or mixtures thereof; or an aliphatic, aromatic or heteroaromatic alcohol, for example alkanols like methanol, ethanol, propanol, isopropanol, n-butanol, isobutanol or tert-butanol or mixtures thereof, preferably alcohol solvent and most preferably ethanol.

In another embodiment, the present invention provides (S)-1-amino-2,3-dihydro-1H-indene-4-carbonitrile di-p-toluyl-L-tartaric acid salt.

In another embodiment, the present invention provides an improved process for the preparation of (S)-1-amino-2,3-dihydro-1H-indene-4-carbonitrile optically active acid salt (IIa).

The reaction comprises, reacting of (R,S)-1-amino-2,3-dihydro-1H-indene-4-carbonitrile or its salt using optically active acid in presence of a solvent to produce (S)-1-amino-2,3-dihydro-1H-indene-4-carbonitrile optically active acid salt (IIa).

The reaction is carried out at temperature below 75° C., preferably below 50° C., more preferably below 35° C. The salt formation can be carried out in a solvent selected from a hydrocarbon, as for example aliphatic or aromatic solvents, like for example pentane, hexane, cyclohexane, heptane, methylcyclohexane (MCH), toluene and xylene etc. or mixtures thereof; or a cyclic or noncyclic ether, like non-cyclic di-C₁-C₄-alkyl ethers, as for example methyl-t-butyl ether, diethyl ether, dibutyl ether, diisopropyl ether, cyclic ethers, like 1,4-dioxane or tetrahydrofuran or mixtures thereof; or a ketone solvent like di-C₁-C₄-alkyl ketones, as for example acetone, butanone, methyl ethyl ketone, methyl isobutyl ketone, diisobutyl ketone, methyl isopropyl ketone or mixtures thereof; or an aliphatic, aromatic or heteroaromatic alcohol, for example alkanols like methanol, ethanol, propanol, isopropanol, n-butanol, isobutanol or tert-butanol or mixtures thereof, preferably alcohol solvent and most preferably ethanol.

In another embodiment, the present invention provides an improved process for the preparation of (S)-1-amino-2,3-dihydro-1H-indene-4-carbonitrile.

The reaction comprises, reacting of (R,S)-1-amino-2,3-dihydro-1H-indene-4-carbonitrile using optically active acid in the presence of solvent to produce diasteromeric salt of (S)-1-amino-2,3-dihydro-1H-indene-4-carbonitrile with optically active acid.

The reaction is carried out at temperature below 75° C., preferably below 50° C., more preferably below 35° C. For the separation via diasteromeric salts, all optically active acids are suitable in principle, such L-tartaric acid, D-tartaric acid, di-p-toluoyl-L-tartaric acid, dibenzoyl tartaric acid, malic acid, mandelic acid, (+)-camphor-10-sulfonic acid etc. Preferably, L-tartaric acids are used and most preferably di-p-toluoyl-L-tartaric acid can be used as optically active acid. The salt formation can be carried out in a solvent selected from a hydrocarbon, as for example aliphatic or aromatic solvents, like for example pentane, hexane, cyclohexane, heptane, methylcyclohexane (MCH), toluene and xylene etc. or mixtures thereof; or a cyclic or noncyclic ether, like non-cyclic di-C₁-C₄-alkyl ethers, as for example methyl-t-butyl ether, diethyl ether, dibutyl ether, diisopropyl ether, cyclic ethers, like 1,4-dioxane or tetrahydrofuran or mixtures thereof; or a ketone solvent like di-C₁-C₄-alkyl ketones, as for example acetone, butanone, methyl ethyl ketone, methyl isobutyl ketone, diisobutyl ketone, methyl isopropyl ketone or mixtures thereof; or an aliphatic, aromatic or heteroaromatic alcohol, for example alkanols like methanol, ethanol, propanol, isopropanol, n-butanol, isobutanol or tert-butanol or mixtures thereof, preferably alcohol solvent and most preferably ethanol.

(S)-1-Amino-2,3-dihydro-1H-indene-4-carbonitrile optically active acid salt is treated with a suitable base in presence of a solvent to produce (S)-1-amino-2,3-dihydro-1H-indene-4-carbonitrile.

The reaction is carried out at a temperature of 25-30° C., preferably 30° C. The reaction is carried out in presence of an organic base selected from the group comprising N-methylmorpholine, triethylamine, diisopropylethylamine, N,N-dimethylpiperazine, pyridine or mixtures thereof; in an organic solvent selected from methylene chloride, ethyl acetate, tetrahydrofuran, N,N-dimethylformamide, toluene, acetonitrile, acetone or mixtures thereof; in an organic solvent selected from a hydrocarbon, for example pentane, hexane, cyclohexane, heptane, methylcyclohexane (MCH), toluene and xylene etc. or mixtures thereof; or an ether, for example methyl-t-butyl ether, diethyl ether, dibutyl ether, diisopropyl ether, 1,4-dioxane or tetrahydrofuran; or a ketone solvent for example acetone, butanone, methyl ethyl ketone, methyl isobutyl ketone, diisobutyl ketone, methyl isopropyl ketone; or an alcohol, for example methanol, ethanol, propanol, isopropanol, butanol or isobutanol, or an ester for example methyl-acetate, ethyl-actetate or isopropyl-acetate, acetonitrile, N,N-dimethylformamide, N-methyl-pyrrolidone, dimethylsulfoxide, or mixtures thereof.

Alternatively, the reaction can be carried out using an inorganic base such as metal hydroxide in water, where metal ion could be Li, Na, K, Cs, Ca, Ba, Mg, Al, Zn ion or any mixture thereof. The mixture can further consist of an organic solvent selected from a hydrocarbon, as for example aliphatic or aromatic solvents, like for example pentane, hexane, cyclohexane, heptane, methylcyclohexane (MCH), toluene and xylene etc. or mixtures thereof; or a cyclic or noncyclic ether, like non-cyclic di-C₁-C₄-alkyl ethers, as for example methyl-t-butyl ether, diethyl ether, dibutyl ether, diisopropyl ether, 1,4-dioxane or tetrahydrofuran; or a ketone solvent like di-C₁-C₄-alkyl ketones, as for example acetone, butanone, methyl ethyl ketone, methyl isobutyl ketone, diisobutyl ketone, methyl isopropyl ketone or mixtures thereof.

After completion of the reaction, reaction mass was filtered and the filtrate was distilled to obtain (S)-1-amino-2,3-dihydro-1H-indene-4-carbonitrile (II).

The route of synthesis is shown in scheme-III:

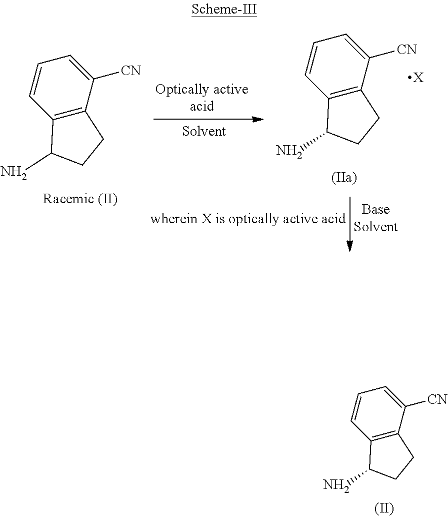

In another embodiment, the present invention also provides a process for the preparation of Ozanimod or its pharmaceutically acceptable salts thereof.

The process comprises, reacting (S)-5-(3-(1-amino-2,3-dihydro-1H-inden-4-yl)-1,2,4-oxadiazol-5-yl)-2-isopropoxy-benzonitrile or its salt (XI) with alkyl-2-haloacetate (XII) in the presence of suitable base and a suitable solvent to produce compound of formula (XIII) (S)-5-(3-(aminoethanoic acid alkyl ester-2,3-dihydro-1H-inden-4-yl)-1,2,4-oxadiazol-5-yl)-2-isopropoxy-benzonitrile. The alkyl-2-haloacetate is selected from ethyl-2-chloroacetate, ethyl-2-bromoacetate, ethyl-2-iodoacetate, methyl-2-chloroacetate, methyl-2-bromoacetate, methyl-2-iodoacetate, isopropyl-2-chloroacetate, isopropyl-2-bromoacetate, isopropyl-2-iodoacetate etc, preferably ethyl-2-chloroacetate.

The reaction is carried out at a temperature of below 110° C., preferably below 80° C. The base used in the above reaction is alkali or alkaline earth metal hydroxide selected from a group comprising lithium hydroxide, sodium hydroxide, potassium hydroxide, cesium hydroxide, calcium hydroxide, barium hydroxide, aluminium hydroxide, magnesium hydroxide, zinc hydroxide; an alkali metal alkoxide selected from a group comprising sodium methoxide, or sodium ethoxide; an alkali metal carbonate selected from a group comprising sodium carbonate or potassium carbonate or cesium carbonate; an alkali metal hydrogencarbonate selected from a group comprising sodium hydrogencarbonate or potassium hydrogencarbonate; or mixtures thereof. The suitable solvent used in the above reaction is selected from a group comprising a hydrocarbon, as for example aliphatic or aromatic solvents, like for example pentane, hexane, cyclohexane, heptane, methylcyclohexane (MCH), toluene and xylene etc. or mixtures thereof; or a cyclic or noncyclic ether, like non-cyclic di-C₁-C₄-alkyl ethers, as for example methyl-t-butyl ether, diethyl ether, dibutyl ether, diisopropyl ether, cyclic ethers, like 1,4-dioxane or tetrahydrofuran; or a ketone solvent like di-C₁-C₄-alkyl ketones, as for example acetone, butanone, methyl ethyl ketone, methyl isobutyl ketone, diisobutyl ketone, methyl isopropyl ketone; or an aliphatic, aromatic or heteroaromatic alcohol, for example alkanols like methanol, ethanol, propanol, isopropanol, butanol or isobutanol, or an ester like C₁-C₄-alkyl esters of a monocarboxylic acid, as for example methyl-acetate, ethyl-actetate or isopropyl-acetate, acetonitrile, N,N-dimethylformamide, N-methyl-pyrrolidone, dimethylsulfoxide, or mixtures thereof.

Further reduction of compound of formula (XIII) (S)-5-(3-(aminoethanoic acid alkyl ester-2,3-dihydro-1H-inden-4-yl)-1,2,4-oxadiazol-5-yl)-2-isopropoxy-benzonitrile is carried out in the presence of reducing agent and an organic solvent. The suitable reducing agent is sodium borohydride optionally in combination with BF₃.etherate etc.

The reaction is carried out in presence of organic solvent comprising a hydrocarbon, as for example aliphatic or aromatic solvents, like for example pentane, hexane, cyclohexane, heptane, methylcyclohexane (MCH), toluene and xylene etc. or mixtures thereof; or a cyclic or noncyclic ether, like non-cyclic di-C₁-C₄-alkyl ethers, as for example methyl-t-butyl ether, diethyl ether, dibutyl ether, diisopropyl ether, cyclic ethers, like 1,4-dioxane or tetrahydrofuran; or a ketone solvent like di-C₁-C₄-alkyl ketones, as for example acetone, butanone, methyl ethyl ketone, methyl isobutyl ketone, diisobutyl ketone, methyl isopropyl ketone; or an aliphatic, aromatic or heteroaromatic alcohol, for example alkanols like methanol, ethanol, propanol, isopropanol, butanol or isobutanol, or an ester like C₁-C₄-alkyl esters of a monocarboxylic acid, as for example methyl-acetate, ethyl-actetate or isopropyl-acetate, acetonitrile, N,N-dimethylformamide, N-methyl-pyrrolidone, dimethylsulfoxide, or mixtures thereof.

In another embodiment, Ozanimod (I) is converted to its pharmaceutically acceptable salt by treating Ozanimod (I) with appropriate acid in presence of a suitable solvent selected from the group comprising methanol, ethanol, isopropanol, tetrahydrofuran, ethyl acetate, acetone, acetonitrile, hexane, heptane, cyclohexane, methylene chloride or mixtures thereof.

Pharmaceutically acceptable salts of Ozanimod (I)) include acid salt selected from the group comprising mineral acid salts selected from hydrochloride, hydrobromide, hydroiodide, sulfates salts, nitrate salts, phosphates salts or perchlorate; organic acid salts selected from acetates, propionates, lactates, fumarates, tartaric acid salts, maleates, mandelates, glutamates, glutarates, citrates salts, ascorbates; gluconates; succinates; sulfonates such as methanesulfonates, benzenesulfonates, or p-toluenesulfonates; and amino acid salts selected from aspartates or glutamates.

The acid is mineral acid selected from the group comprising hydrochloride, hydrobromide, hydroiodide, sulfuric acid, nitric acid, phosphoric acid, carbonic acid, or perchloric acid; organic acid selected from the group of mono- or polycarboxylic acids, in particular mono-, di- or tricarboxylic acids, comprising acetic acid, propionic acid, lactic acid, maleic acid, fumaric acid, tartaric acid, malic acid, citric acid, ascorbic acid; sulfonic acid selected from the group comprising methanesulfonic acid, benzenesulfonic aid, or p-toluenesulfonic acid; and acidic amino acid selected from the group comprising aspartic acid or glutamic acid or mixtures thereof.

The route of synthesis is shown in scheme-IV:

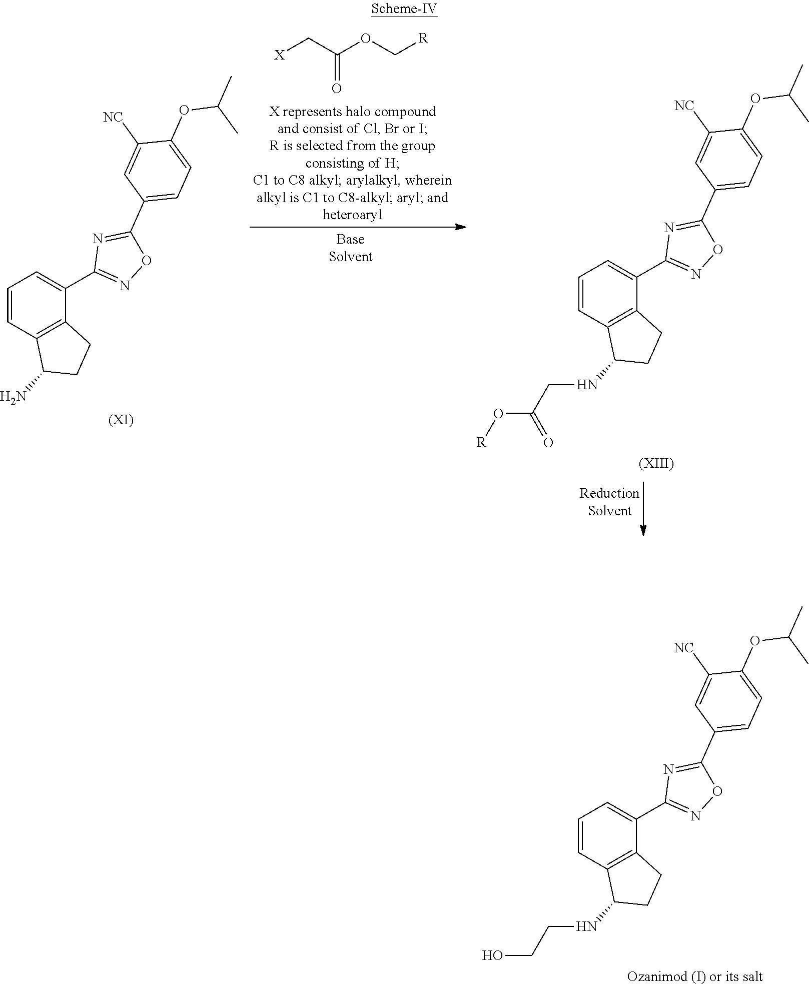

The following examples illustrate the nature of the invention and are provided for illustrative purposes only and should not be construed to limit the scope of the invention.

### EXAMPLES

### Example-1

### Process for the Preparation of Ozanimod

Step-i:

A mixture of ethyl chloroacetate (1.5 moles) and acetonitrile (10.0 volumes) was added to the mixture of (S)-5-(3-(1-amino-2,3-dihydro-1H-inden-4-yl)-1,2,4-oxadiazol-5-yl)-2-isopropoxy-benzonitrile, Na₂CO₃ (2.5 moles). The resulting reaction mixture was heated to 75-85° C. for reaction completion. The product was extracted with ethyl acetate and distilled off completely. To the crude material MTBE (5.0 volumes) was added at 25-35° C. and stirred for 1.0 hr. The solid was filtered, washed with MTBE and dried to yield compound (S)-5-(3-(aminoethanoic acid ethyl ester-2,3-dihydro-1H-inden-4-yl)-1,2,4-ox adiazol-5-yl)-2-isopropoxy-benzonitrile.

Step-ii:

Sodium borohydride (3.0 moles) was added to the suspension of compound of (S)-5-(3-(aminoethanoic acid ethyl ester-2,3-dihydro-1H-inden-4-yl)-1,2,4-oxadiazol-5-yl)-2-isopropoxy-benzonitrile in ethanol (10 volumes) at 25-35° C. and maintained the reaction for 2 hrs at 75-85° C. After completion of reaction 10% aqueous acetic acid (10 volumes) and MTBE were added to the reaction mixture at 25-35° C. The pH of the aqueous layer was adjusted to about 9.0 with 8% aq. K₂CO₃ solution. The reaction mass was stirred for 1.0 hr and filtered the solid material. Wet solid was treated with acetonitrile at 25-35° for 1.0 hr. The solid was filtered and further treated with toluene to afford Ozanimod.

¹H-NMR (500 MHz, DMSO-d₆) δ (ppm):

δ 8.47 (d, J:2.8 Hz, 1H), δ 8.38 (dd, J:9.0 Hz, 1H), δ 7.96 (d, J:7.68 Hz, 1H), δ 7.54 (m, 2H), δ 7.40 (t, J:7.6 Hz, 1H), δ4.96 (m, 1H), δ 4.53 (s, 1H), δ 4.22 (t, J:6.5 Hz, 1H), δ 3.49 (t, J:5.5 Hz, 2H), δ 3.31 (td, J:8.6 Hz, 1H), δ 3.05 (m, 1H), δ 2.67 (td, J:12.2, J2: 6.2 Hz, 2H), 2.36 (dq, J:12.1, J2: 3.7 Hz, 1H), δ 2.01 (s, 1H), δ 1.82 (m, 1H), δ 1.38 (d, J:6.2 Hz, 6H).

¹³C-NMR (125 MHz, DMSO-d₆) δ (ppm):

δ 172.9, 168.3, 162.4, 174.5, 142.9, 134.4, 133.6, 127.1, 126.7, 122.3, 115.9, 115.2, 114.7, 102.4, 72.5, 62.4, 60.9, 49.2, 32.6, 31.4, 21.5.

### Example-2

### Process for the Preparation of Ozanimod

Step-i:

A mixture of ethyl chloroacetate (1.5 moles) and DMF (10.0 volumes) was added to the mixture of (S)-5-(3-(1-amino-2,3-dihydro-1H-inden-4-yl)-1,2,4-oxadiazol-5-yl)-2-isopropoxy-benzonitrile, Na₂CO₃ (2.5 moles). The resulting reaction mixture was heated to 75-85° C. for reaction completion. The product was extracted with ethyl acetate and distilled off completely. To the crude material MTBE (5.0 volumes) was added at 25-35° C. and stirred for 1.0 hr. The solid was filtered, washed with MTBE and dried to yield compound (S)-5-(3-(aminoethanoic acid ethyl ester-2,3-dihydro-1H-inden-4-yl)-1,2,4-ox adiazol-5-yl)-2-isopropoxy-benzonitrile.

Step-ii:

Sodium borohydride (3.0 moles) was added to the suspension of compound of (S)-5-(3-(aminoethanoic acid ethyl ester-2,3-dihydro-1H-inden-4-yl)-1,2,4-ox adiazol-5-yl)-2-isopropoxy-benzonitrile in ethanol (10 volumes) at 25-35° C. and maintained the reaction for 2 hrs at 75-85° C. After completion of reaction 10% aqueous acetic acid (10 volumes) and MTBE were added to the reaction mixture at 25-35° C. The pH of the aqueous layer was adjusted to about 9.0 with 8% aq. K₂CO₃ solution. The reaction mass was stirred for 1.0 hr and filtered the solid material. Wet solid was treated with acetonitrile at 25-35° for 1.0 hr. The solid was filtered and further treated with IPA to afford Ozanimod.

### Example-3

### Process for the Preparation of Ozanimod

Step-i:

A mixture of ethyl chloroacetate (1.5 moles) and DMPU (dimethyl propylene urea) (10.0 volumes) was added to the mixture of (S)-5-(3-(1-amino-2,3-dihydro-1H-inden-4-yl)-1,2,4-oxadiazol-5-yl)-2-isopropoxy-benzonitrile, Na₂CO₃ (2.5 moles). The resulting reaction mixture was heated to 75-85° C. for reaction completion. The product was extracted with ethyl acetate and distilled off completely. To the crude material MTBE (5.0 volumes) was added at 25-35° C. and stirred for 1.0 hr. The solid was filtered, washed with MTBE and dried to yield compound (S)-5-(3-(aminoethanoic acid ethyl ester-2,3-dihydro-1H-inden-4-yl)-1,2,4-oxadiazol-5-yl)-2-isopropoxy-benzonitrile.

Step-ii:

Sodium borohydride (3.0 moles) was added to the suspension of compound of (S)-5-(3-(aminoethanoic acid ethyl ester-2,3-dihydro-1H-inden-4-yl)-1,2,4-oxadiazol-5-yl)-2-isopropoxy-benzonitrile in ethanol (10 volumes) at 25-35° C. and maintained the reaction for 2 h at 75-85° C. After completion of reaction 10% aqueous acetic acid (10 volumes) and MTBE were added to the reaction mixture at 25-35° C. The pH of the aqueous layer was adjusted to about 9.0 with 8% aq. K₂CO₃ solution. The reaction mass was stirred for 1.0 hr and filtered the solid material. Wet solid was treated with acetonitrile at 25-35° for 1.0 hr. The solid was filtered and further treated with acetone to afford Ozanimod.

### Example-4

### Process for the Preparation of (S)-1-amino-2,3-dihydro-1H-indene-4-carbonitrile di-p-toluoyl-L-tartaric acid (DTTA) salt

Di-p-toluoyl-L-tartaric acid (0.25 moles) was added to the solution of (RS) 1-amino-2,3-dihydro-1H-indene-4-carbonitrile (10 gm) in IPA (10 volumes) at 25-30° C. The reaction mass was stirred for 4.0 hrs at 25-35° C. and further cooled to 0-10° C., maintained for 30 mints. Filtered the solid and washed with IPA to afford (S)-1-amino-2,3-dihydro-1H-indene-4-carbonitrile di-p-toluoyl-L-tartaric acid (DTTA) salt.

### Example-5

### Process for the Preparation of (S)-1-amino-2,3-dihydro-1H-indene-4-carbonitrile di-p-toluoyl-L-tartaric acid (DTTA) salt

Di-p-toluoyl-L-tartaric acid (0.25 moles) was added to the solution of (RS) 1-amino-2,3-dihydro-1H-indene-4-carbonitrile (10 gm) in ethyl acetate (10 volumes) at 25-30° C. The reaction mass was stirred for 4.0 hrs at 25-35° C. and further cooled to 0-10° C., maintained for 30 mints. Filtered the solid and washed with ethyl acetate to afford (S)-1-amino-2,3-dihydro-1H-indene-4-carbonitrile di-p-toluoyl-L-tartaric acid (DTTA) salt.

### Example-6

### Process for the Preparation of (S)-1-amino-2,3-dihydro-1H-indene-4-carbonitrile (II)

Step-i:

### Preparation of (S)-1-amino-2,3-dihydro-1H-indene-4-carbonitrile di-p-toluoyl-L-tartaric acid (DTTA) salt

Di-p-toluoyl-L-tartaric acid (0.25 moles) was added to the solution of (RS) 1-amino-2,3-dihydro-1H-indene-4-carbonitrile (10 gm) in ethanol (10 volumes) at 25-30° C. The reaction mass was stirred for 4.0 hrs at 25-35° C. and further cooled to 0-10° C., maintained for 30 mints. Filtered the solid and washed with ethanol. The obtained crude product was treated with ethanol (10 volumes) at 75-85° C. for 30 mints and further cooled to 0-10° C. and maintained for 1.0 hr. Filtered the solid and washed with ethanol to afford (S)-1-amino-2,3-dihydro-1H-indene-4-carbonitrile di-p-toluoyl-L-tartaric acid (DTTA) salt. (Yield: 65.7%, Purity by HPLC: 99%)

Step-ii:

Triethylamine was added to a solution of (S)-1-amino-2,3-dihydro-1H-indene-4-carbonitrile di-p-toluoyl-L-tartaric acid (DTTA) salt in DCM at 25-30° C. The reaction mass was stirred for 2 hrs and separated the layers. The DCM layer was distilled off to afford (S)-1-amino-2,3-dihydro-1H-indene-4-carbonitrile.

1-17. (canceled)

18. An optically active acid salt of (S)-1-amino-2,3-dihydro-1H-indene-4-carbonitrile, the salt having a formula (IIa): 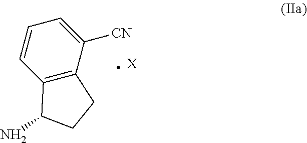
wherein X is an optically active acid selected from the group consisting of L-tartaric acid, D-tartaric acid, di-p-toluoyl-L-tartaric acid, dibenzoyl tartaric acid, malic acid, mandelic acid, and (+)-camphor-10-sulfonic acid.

19. The optically active acid salt of claim 18, which is a di-p-toluoyl-L-tartaric acid salt of (S)-1-amino-2,3-dihydro-1H-indene-4-carbonitrile having a formula (IIb):

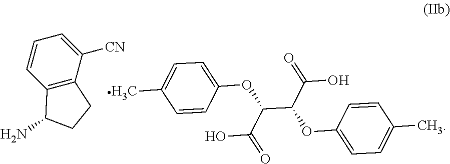 [SMILES: N#Cc1cccc2c1CC[C@@H]2N ; Cc1ccc(O[C@@H](C(=O)O)[C@@H](Oc2ccc(C)cc2)C(=O)O)cc1]

20. A process for the preparation of (S)-1-amino-2,3-dihydro-1H-indene-4-carbonitrile optically active acid salt of formula (IIa), the process comprising:
(a) treating (R,S)-1-amino-2,3-dihydro-1H-indene-4-carbonitrile in a solvent with an optically active acid to form the optically active acid salt of formula (IIa); and
(b) filtering the optically active acid salt of formula (IIa).

21. The process according to claim 20, wherein the solvent is selected from the group consisting of hexane, cyclohexane, benzene, toluene, methyl-t-butyl ether, diethyl ether, dibutyl ether, tetrahydrofuran, acetone, butanone, methyl ethyl ketone, methyl isobutyl ketone, diisobutyl ketone, methyl isopropyl ketone, ethyl acetate, acetone, methanol, ethanol, propanol, isopropanol, butanol, isobutanol, and mixtures thereof.

22. The process according to claim 20, wherein the solvent is ethanol.

23. The process according to claim 20, wherein the optically active acid is selected from the group consisting of L-tartaric acid, D-tartaric acid, di-p-toluoyl-L-tartaric acid, dibenzoyl tartaric acid, malic acid, mandelic acid, and (+)-camphor-10-sulfonic acid.

24. The process according to claim 20, wherein the optically active acid is di-p-toluoyl-L-tartaric acid.

25. A process for the preparation of (S)-1-amino-2,3-dihydro-1H-indene-4-carbonitrile having a formula (II), 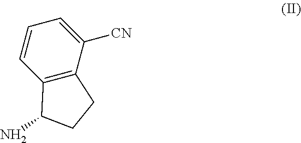 [SMILES: N#Cc1cccc2c1CC[C@@H]2N]
the process comprising:
(a) treating (R,S)-1-amino-2,3-dihydro-1H-indene-4-carbonitrile in a solvent with an optically active acid to form an optically active acid salt of the (S)-1-amino-2,3-dihydro-1H-indene-4-carbonitrile having the formula (II);
(b) filtering the optically active acid salt of the (S)-1-amino-2,3-dihydro-1H-indene-4-carbonitrile; and
(c) converting the optically active acid salt of the (S)-1-amino-2,3-dihydro-1H-indene-4-carbonitrile to (S)-1-amino-2,3-dihydro-1H-indene-4-carbonitrile in the presence of a solvent and a base.

26. The process according to claim 25, wherein the optically active acid is selected from the group consisting of L-tartaric acid, D-tartaric acid, di-p-toluoyl-L-tartaric acid, dibenzoyl tartaric acid, malic acid, mandelic acid, and (+)-camphor-10-sulfonic acid.

27. The process according to claim 25, wherein in step (a) and (c), the solvent is selected from the group consisting of hexane, cyclohexane, benzene, toluene, methyl-t-butyl ether, diethyl ether, dibutyl ether, tetrahydrofuran, acetone, butanone, methyl ethyl ketone, methyl isobutyl ketone, diisobutyl ketone, methyl isopropyl ketone, ethyl acetate, acetone, methanol, ethanol, propanol, isopropanol, butanol, isobutanol, and mixtures thereof.

28. The process according to claim 25, wherein the solvent is ethanol.

29. The process according to claim 25, wherein the base is selected from the group consisting of lithium hydroxide, sodium hydroxide, potassium hydroxide, cesium hydroxide, calcium hydroxide, barium hydroxide, aluminium hydroxide, magnesium hydroxide, zinc hydroxide, sodium methoxide, sodium ethoxide, sodium carbonate, potassium carbonate, cesium carbonate, sodium hydrogen carbonate, potassium hydrogen carbonate, and mixtures thereof.

30. The process according to claim 25, wherein the base is sodium hydroxide.

31. A process for the preparation of Ozanimod having a formula (I), or its pharmaceutically acceptable salts, 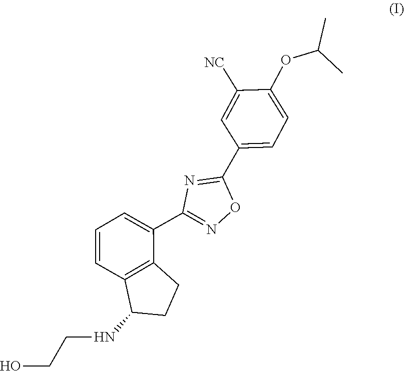 [SMILES: CC(C)Oc1ccc(-c2nc(-c3cccc4c3CC[C@@H]4NCCO)no2)cc1C#N]
the process comprising:
(i) condensing (S)-5-(3-(1-amino-2,3-dihydro-1H-inden-4-yl)-1,2,4-oxadiazol-5-yl)-2-isopropoxy-benzonitrile (XI) or a salt thereof, 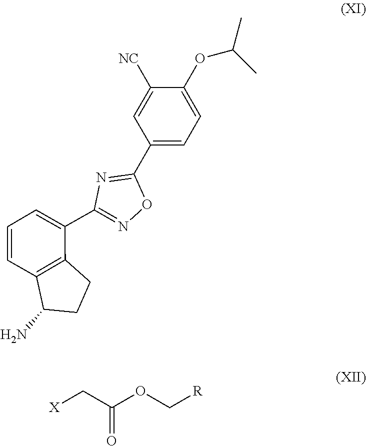
with a 2-haloacetate of formula (XII),
wherein:
X represents a halo compound selected from the group consisting of Cl, Br or I;
R is selected from the group consisting of H, C₁ to C₈ alkyl, arylalkyl, wherein the alkyl is C₁ to C₈ alkyl, aryl, and heteroaryl;
in the presence of a base and a solvent, to produce (S)-5-(3-(aminoethanoic acid alkyl ester-2,3-dihydro-1H-inden-4-yl)-1,2,4-oxadiazol-5-yl)-2-isopropoxy-benzonitrile (XIII); 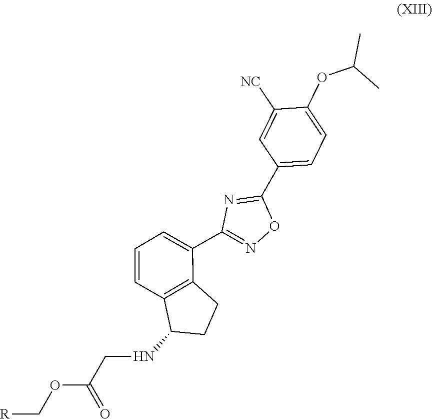
(ii) reducing the (S)-5-(3-(aminoethanoic acid alkyl ester-2,3-dihydro-1H-inden-4-yl)-1,2,4-oxadiazol-5-yl)-2-isopropoxy-benzonitrile (XIII) in the presence of a solvent to produce Ozanimod (I); and
(iii) optionally, converting Ozanimod (I) to a pharmaceutically acceptable salt, or a hydrate or solvate thereof.

32. The process according to claim 31, wherein the 2-haloacetate of formula (XII) is selected from the group consisting of ethyl-2-chloroacetate, ethyl-2-bromoacetate, ethyl-2-iodoacetate, methyl-2-chloroacetate, methyl-2-bromoacetate, methyl-2-iodoacetate, isopropyl-2-chloroacetate, isopropyl-2-bromoacetate, and isopropyl-2-iodoacetate.

33. The process according to claim 31, wherein the solvent in step (i) and step (ii) is selected from the group consisting of hexane, cyclohexane, benzene, toluene, acetonitrile, dimethyl propylene urea (DMPU), dimethylformamide (DMF), methyl-t-butyl ether, diethyl ether, dibutyl ether, tetrahydrofuran, acetone, butanone, methyl ethyl ketone, methyl isobutyl ketone, diisobutyl ketone, methyl isopropyl ketone, methanol, ethanol, propanol, isopropanol, butanol, isobutanol, and mixtures thereof

34. The process according to claim 31, wherein the solvent is acetonitrile.

35. The process according to claim 31, wherein the base in step (i) is selected from the group consisting of lithium hydroxide, sodium hydroxide, potassium hydroxide, cesium hydroxide, calcium hydroxide, barium hydroxide, aluminium hydroxide, magnesium hydroxide, zinc hydroxide, sodium methoxide, sodium ethoxide, sodium carbonate or potassium carbonate, cesium carbonate, sodium hydrogen carbonate, potassium hydrogen carbonate, and mixtures thereof.

36. The process according to claim 31, wherein the base is sodium carbonate.

37. The process according to claim 31, wherein the reducing agent is selected from the group consisting of sodium borohydride and sodium borohydride in combination with BF₃ etherate.
